(19) United States
(12) Patent Application Publication
(10) Pub. No.: US 2010/0065576 A1
(43) Pub. Date: Mar. 18, 2010

(54) Device for Holding and Controlled Dispensing of at Least Partially Porous Absorption Bodies and an Absorption Body for Use Therewith

(75) Inventor: Robert Johannes Verheij, Rotterdam (NL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cameron of Lochiel B.V. a corporation

(21) Appl. No.: 12/516,882

(22) PCT Filed: Nov. 26, 2007
(86) PCT No.: PCT/NL2007/050593

§ 371 Date: Sep. 15, 2009

(30) Foreign Application Priority Data
Nov. 30, 2006 (NL) 1032972

(51) Int. Cl. B65D 83/02; A61F 13/15

(52) U.S. Cl. 221/42; 604/385.01

(57) ABSTRACT

A device for retaining and dispensing in a controlled fashion at least partially porous absorbent pads (20) that includes an elongated storage chamber (11) that is intended and adjusted to receive inside a series of absorbent pads and that includes actuators (30) that are capable and have been set up to move an absorbent pad in the storage chamber to an exit opening (13) and dispense it at the exit opening. The actuators comprise a manually controllable control instrument (31, 32) that is capable and adjusted to enforce rotation on the carrier bodies and to directly hold on to them as well as a spiral profile (14) over at least nearly the full length of an internal wall (12) of the storage chamber (11). The absorbent pads comprise holding means (23) that are capable and have been set up to work together with the spiral profile (14) in the storage chamber so that a passageway can be enforced according to the spiral profile (14) for an absorbent pad with regard to rotation imposed by the control instrument.

The present invention relates to a device for retaining and dispensing in a controlled fashion at least partially porous absorbent pads that includes an elongated storage chamber that is intended and adjusted to receive inside a series of absorbent pads and that includes actuators that are capable and have been set up to move an absorbent pad in the storage chamber to an exit opening and dispense it at the exit opening whereby the actuators include a manually controlled control mechanism that is capable of and adjusted to enforce a rotation on the carrier bodies. The invention also relates to an absorbent pad to be used with such a device that at least includes a basic part that in effect retains its shape that has been provided on top with a porous absorption layer.

The absorbent pads usually include a basic pad that retains its shape with on top a porous, cotton wool type absorption layer for the absorption of or for dispensing substances, in particular, cosmetic and pharmaceutical products. The device is meant for retaining a stack of such absorbent pads and to dispense the pads as required. Such a device is known from the U.S. Pat. No. 4,854,761 description and includes a tubular holder provided with a storage chamber for a series of absorbent pads. The storage chamber is provided with actuators that are capable and have been set up to drive the absorbent pads in the storage chamber to the exit opening. The actuators include a spring construction that has been added to one of the exit opening sides that has been turned away from the absorbent pads and that exercises an axial pressure on the freely prevalent axial carrier bodies. The holder is also provided with a control instrument that holds one of the rear absorbent pad through a spring construction to rotate it while consecutive absorbent pads are being held together. The holder is provided on the front with an end section that has been provided on the inside with a spiral profile in the form of a thread to receive on this a complementary groove of a foremost absorbent pad. Whilst turning, the foremost absorbent pad will be placed in the end-piece on the spiral profile to be guided forwards over this while it is being kept rotational by the next absorbent pad and ultimately it leaves the holder.

A disadvantage of such a device is, however, that the required spring construction and the required end-piece comprise an important part of the length of the device and, therefore, limit the effective supply capacity. Moreover, the use of the common device for common cosmetic and pharmaceutical applications, in particular, is insufficiently hygienic because the foremost absorbent pad protrudes from the holder to a relatively small extent and thus inevitably one of the edges of the exit opening can come into contact with the skin or a part of the surface to be treated when the holder is used.

The object of the present invention is to provide a device of the type described in the opening lines that allows that the storage chamber can be extended over practically the whole length thereof as well as other objectives. Another object of the present invention is to ensure that a more hygienic use can be achieved through an absorbent pad to be used therewith.

To achieve the first object, a device of the type described in the opening lines has as a property that the actuators comprise a spiral profile over at least nearly a full length of an inside wall of the storage chamber, that the spiral profile is capable and adjusted to be in the storage chamber with absorbent pads provided with holding means as an assembly that works together through rotation enforced on it by the control instrument to enforce a passageway according to the spiral profile and that the control instrument is capable and adjusted to directly hold the absorbent pads according to the invention. The spiral profile is already in the storage chamber so that the actuators can already work together in the storage chamber with the absorbent pads and a separate end-piece can be avoided. Since the control instrument directly holds the absorbent pads, a spring construction or similar is, moreover, not needed as is required with the common device and the device is, thus, available for virtually the full length for holding a stack of absorbent pads.

In a particular modification, the device has, according to the invention, as a property that the spiral profile comprises a groove that has been added to the inside wall and that the holding means at least comprise one wing instrument that goes out to one side of an absorbent pad and is capable and adjusted to enter into the spiral profile. Since at least one wing instrument enters into the groove, an absorbent pad will be forced in a forward movement if thereafter rotation is enforced using the control instrument that will ultimately appear at the exit opening.

The device according to the invention has, in a preferred embodiment, as a property that the control instrument comprises a rotation instrument on one of the ends that has been turned away on one of the exit openings of the holder whereof at least one elongated propel instrument protrudes that extends over at least nearly the full length of the storage chamber at least nearly in parallel to a spiral axis of the spiral profile and that is capable and adjusted to hold the absorbent pads. Since the propel instrument, therefore, extends over practically the full length of the storage chamber, the propel instrument can directly hold all absorbent pads and move these forward individually. The absorbent pads working together as is the case with the common device is, therefore, not required nor is compression as provided by a spring construction.

The propel instrument can extend axially according to the spiral axis whereby the absorbent pads have similarly been provided with a passageway opening; yet in a further preferred embodiment the device according to the invention is characterised by the fact that at least one propel instrument will extend between the stored absorbent pads and the inside wall of the storage chamber. The one or more propel instruments will, therefore, be adjacent to the absorbent pads that, in turn, can be closed in favour of an optimal use surface.

The propel instrument can in itself hold the absorbent pads, that may have been adjusted for this purpose, at any place; the device according to the invention is characterised by the fact that the propel instrument is capable and adjusted to hold the holding means of the absorbent pads in a further preferred embodiment type. Thus, the holding means fulfil a double function, that is, on the one hand, holding the spiral profile so that the absorbent pad is moved forward when rotation takes place according to said spiral profile and, on the other hand, to enforce rotation on an absorbent pad in collaboration with the propel instrument.

To ensure the rotation of the control instrument on the absorbent pads is stable, a further preferred embodiment of the device according to the invention has as a property that the absorbent pads each comprise a number of holding instruments that have been regularly distributed over the outline of the absorbent pad and that the control instrument comprises a similar number of propel instruments that are capable and have been set up to hold the holding instruments and that extend to at least nearly fit between the holding instruments. In particular, that the actuators comprise three propel instruments that exit thereof with a mutual angle of approximately 120 degrees with regard to the spiral axis. The fit of the propel instruments between the holding instruments, thus, avoids a dead twist when the rotation direction is changed and when deploying a rotation.

A further particular modification of the device has as a property according to the invention that a resistance element has been included in a section of the holding means near the exit opening. The resistance element creates a perceptible unloading moment when the absorbent pad is released from the storage chamber and also stops a successive absorbent pad from exiting the spiral profile. This means that the carrier elements are discernibly dispensed one by one to the exit opening. Such a resistance element can be provided in different ways, yet, in a particular modification, the device is characterised according to the invention because the resistance element at least comprises a part of a peripheral edge that extends internally in the exit opening and behind which a carrier element hooks on at least temporarily before exiting through the exit opening.

To ensure that the storage chamber and, in particular, the absorbent pads contained therein, cannot be contaminated and, possibly, dry out, a further preferred embodiment of the device has according to the invention as a property that the exit opening can be sealed by using a sealing cap. If used with liquid impregnated absorbent pads, in particular, the preferred option is that the sealing cap can seal hermetically, that is, that it is liquid- and vapour-tight, to prevent premature drying out.

An aesthetically particularly attractive and, moreover, particularly practical embodiment of the device according to the invention has as a property that the sealing cap, the holder body and the control instrument are at least externally cylindrical with a common external diameter. The device will, in such a case, have a pen-like design that is both ergonomically and visually beneficial. A further preferred embodiment of the device according to the invention has as a property that at least the control instrument can be recognised as such. The control instrument will, thus, be profiled or coloured in a different manner to ensure it can be recognised as such, which will increase user-friendliness. The sealing cap can also be provided to be recognised as such if required.

An absorbent pad of the type described in the opening lines has according to the invention as a property that the absorbent pad has holding means in the basic part that are capable and have been set up to work together with a spiral profile on the inside of the device to allow a more hygienic use of the device. Thus the absorbent pad comprises the holding means in the basic part instead of in the absorption layer as is the case with the common absorbent pad, which ensures that the absorption layer can protrude outside the device while the holding means are still working together with the spiral profile. This will ensure that unintentional contact between an edge of an exit opening of the device and skin or a surface to be treated can be avoided. The absorbent pad is, therefore, in particular, suitable for cosmetic and pharmaceutical applications where such hygiene is of essential importance.

In a further modified embodiment, the absorbent pad is, therefore, characterised according to the invention by the fact that the absorption layer holds an active liquid. The absorbent pad, or at least the absorption layer, is soaked, impregnated or humidified in advance with an active liquid, in particular, of a pharmaceutical or cosmetic nature such as, for example, nail polish remover, a dental bleaching agent, make-up or an anti-insect bite product. Such an absorbent pad with the aforementioned active substance will always be immediately offered for use by the device while a stack can always be driven in a practical manner.

The absorbent pad according to the invention has as a property in a preferred embodiment that the basic part has been provided on the bottom with holding means and that it is limited between the bottom and the top by an extended side wall. The side wall, thus, creates additional in-between space between the holding means on the bottom of the basic part and the absorption layer provided that can be found on the opposite side on top so that said absorption layer can extend over a sufficient distance outside the device when used to avoid contact between the device and a surface to treat.

A further preferred embodiment of the absorbent pad according to the invention has as a property that the basic part comprises a body that is open on the bottom to guarantee an increased storage capacity of which a hollow between the bottom and the top is limited by the raised side wall and that the absorbent pad can be nested with a similar absorbent pad and, in particular, that the side wall runs or, at least, protrudes conically for at least a section of its height. A significant side wall height can be used by nesting the absorbent pads together without damaging (too much) the number of absorbent pads that can be held in the storage chamber of the device. A further modified embodiment of the absorbent pad according to the invention has as a property that the side wall comprises a ridge at the bottom that is capable and adjusted to receive thereon a similar ridge of an absorbent pad stacked on this and has been provided with the holding means where the ridge shall ensure that the holding means are also capable of working together with the spiral profile in the storage chamber when nested together.

A further preferred embodiment of the absorbent pad according to the invention has as a property that the ridge has a height that is greater than the thickness of the absorption layer to ensure that the impregnated absorption layer is squeezed in the device as a result of axial pressure on the absorbent pads themselves. Such a ridge height in relation to the thickness of the absorption layer provides sufficient spacing between the absorption layer and the roof of the hollow in a successive absorbent pad to avoid axial pressure on the absorption layer during use.

A further preferred embodiment of the absorbent pad has as a property that the side wall seals vapour-tight on the ridge to counteract depletion as a result of evaporation or some other form of drying out. With the exception of the foremost absorbent pad in a mutually nested stack, every absorption layer will, thus, be enclosed vapour-tight in a hollow of a successive absorbent pad to ensure that the absorption layer cannot dry out prematurely. The foremost absorbent pad can be protected against drying out in a comparable manner by using a vapour-tight sealing cap or a different sealing method for the storage chamber of the device.

The invention shall now be further explained based on a embodiment example and a related drawing. The following is shown in the drawing:

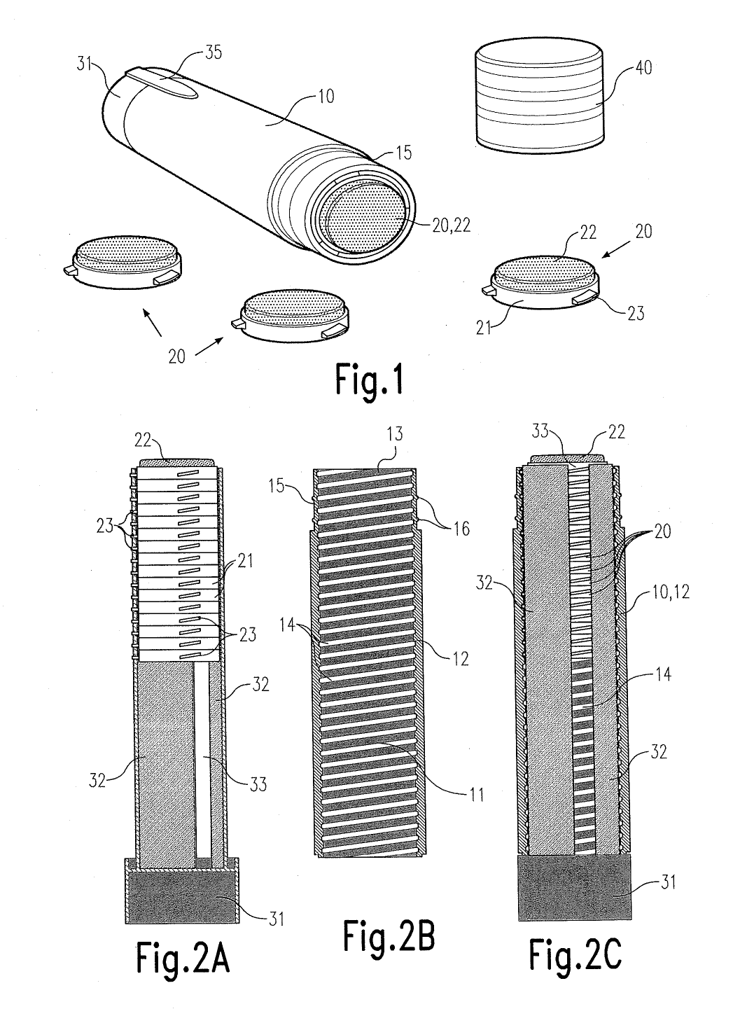

FIG. 1 Shows a perspective view of a first embodiment of a device and absorbent pad according to the invention;

FIG. 2A Shows an exploded view of the actuators of the device of FIG. 1;

FIG. 2B Shows an exploded view of a storage chamber of the device of FIG. 1;

FIG. 2C Shows an exploded view of an assembly of the actuators and storage chamber of FIGS. 2A and 2B;

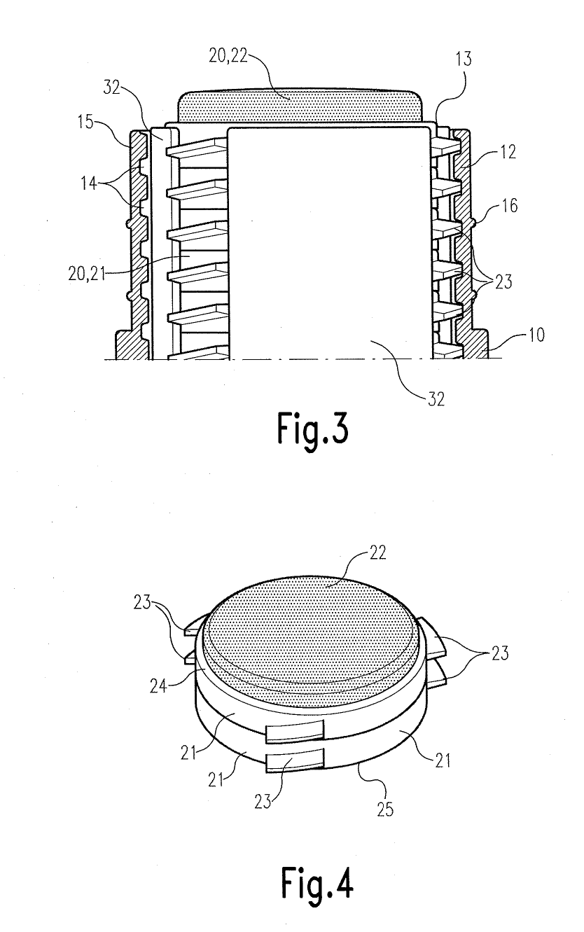

FIG. 3 Shows an enlarged view of an end of the device of FIG. 2C;

FIG. 4 Shows an enlarged perspective view of a first embodiment example of an absorbent pad according to the invention as applied with the device of FIG. 1;

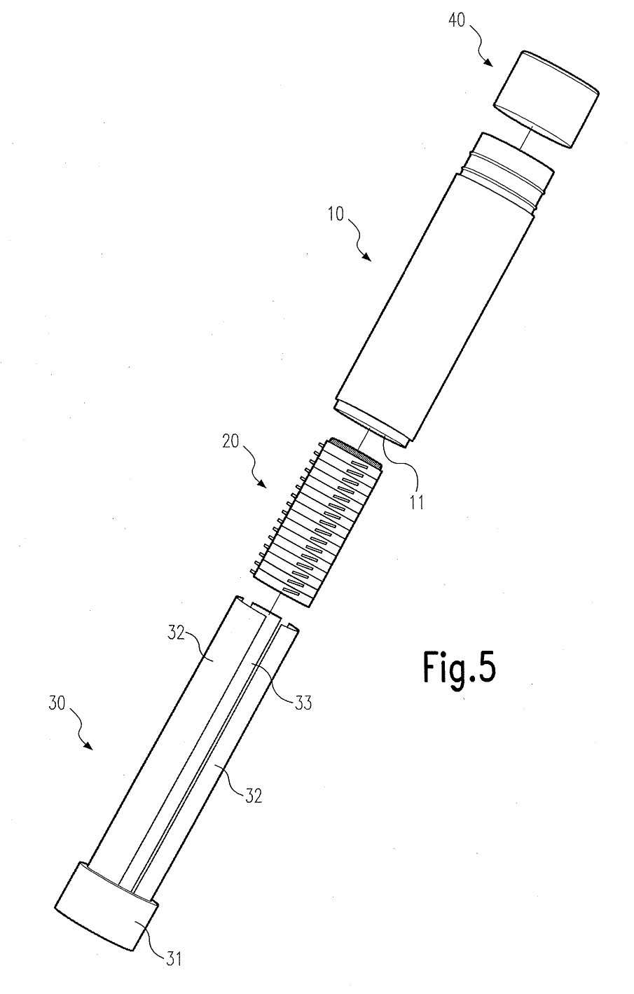

FIG. 5 Shows an exploded drawing of the device of FIG. 1;

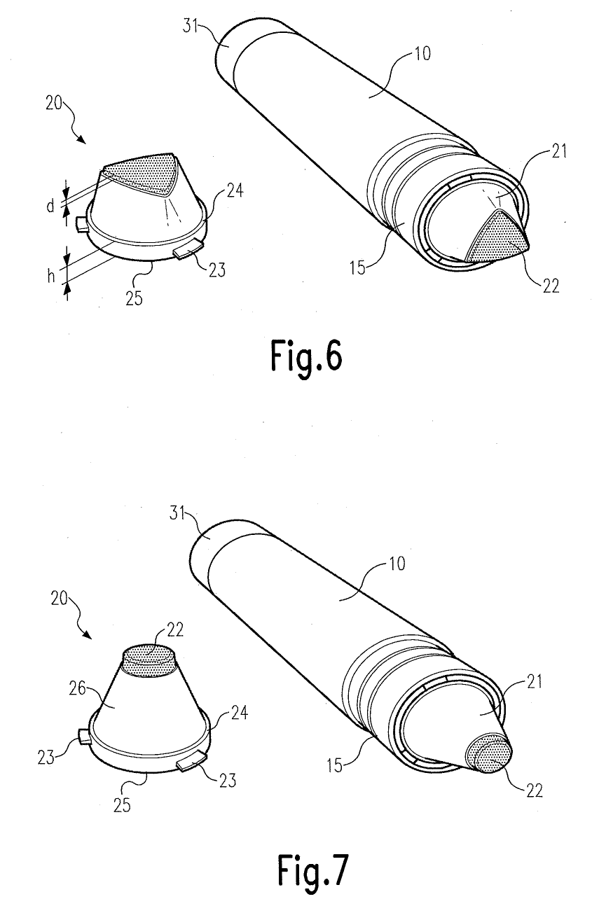

FIG. 6 Shows a perspective view of a second embodiment of a device and absorbent pad according to the invention; and

FIG. 7 Shows a perspective view of a third embodiment of a device and absorbent pad according to the invention. The figures are only schematic and not always drawn using the same scale. In particular, some dimensions may be shown in an exaggerated fashion to a higher or lesser extent for the sake of clarity. Corresponding parts are indicated in the figures with the same reference numbers.

The device of FIG. 1 comprises a refillable holder that looks like a felt-tip pen 10 that comprises an elongated storage chamber 11 over nearly the full length filled with a series of absorbent pads 20. The absorbent pads comprise a plastic basic part that relatively retains its shape 21 that has been provided at the top with a cotton wool type-porous absorption layer 22, also see FIGS. 2 and 3, that may, if required, be soaked in an active liquid or may have been provided with an active substance. The foremost absorbent pad 20 protrudes at the end 15 of the holder 10 by at least the absorption layer 22 through an exit opening 13 that has been provided there and is, therefore, available for use. A sealing cap 40 provided for this can be applied over the end 15 and will hermetically seal the storage chamber 11 and protects the foremost absorbent pad 20. A threaded cap has been assumed in this example that can be screwed on to an external thread 16 provided for this purpose on the end 15. Instead, a sliding cap can, for example, be assumed that is slid on to a flat end 15.

The device can be used for cosmetics such as, for example, applying or removing make-up, nail polish and similar, for pharmaceutical applications such as treating skin conditions or teeth with a suitable active pharmaceutical product or the local sterilisation or sealing of, for example, skin and for a series of other goals such as cleaning, polishing and/or degreasing jewelry, lens or display surfaces and other objects that may or may not be delicate, for the application of adhesive, ointments, wax or ink and for removing stains. The absorption layer is drenched in a substance suitable for the application in advance and, therefore, immediately ready for use with regard to many of these applications.

The device has been provided with manually controllable actuators 30 to move an absorbent pad from the storage chamber 11 to the exit opening 13. The actuators comprise a control instrument 31 in combination with a spiral profile 14 that is mounted over at least nearly the full length of an internal wall 12 of the storage chamber 11. The spiral profile 14 may have been provided in different ways, for example, a spiral-shaped fin or screw thread on the wall 12 or, as in this example, a spiral groove in the wall 12. The absorbent pads 20 are provided with holding means in the form of three wing instruments 23 that protrude laterally from the basic part 21 to enter in the spiral profile 14 when placed in the holder. One position of the wing instruments 23 is harmonised on a pitch of the spiral profile 14 to promote optimal guidance in the spiral profile. The wing instruments 23 are regularly distributed with an angle of approximately 120 degrees among them over an external outline of the basic part 21 to obtain stable guidance of the absorbent pads 20 in the spiral profile 14.

The control instrument 31 comprises a rotary knob that has been included in the holder 10 as a rotational mechanism and can be recognised as such due to a different colour combination, profiling and/or, as shown here, the presence of an attachment clip that looks like a felt-tip pen 35. Three elongated pushing instruments 32 extend from the rotary knob that enter into the storage chamber and maintain among themselves a space 33 to receive in a nearly fitting manner the wing instruments 23 of the absorbent pads 20 there. If the rotary instrument 31 is turned, it will directly hold on to the wing instruments 23 to carry the absorbent pads 20 in their rotation. The wing instruments 23 fulfil within this context a double function by working together with the spiral profile 14 and thus imposing a passageway on to the absorbent pads 20 to that profile 14. Therefore, absorbent pads 20 can be twisted out from the storage chamber 11 according to the pitch of the spiral profile 14 to eventually leave the holder 10 at the exit opening 13. Accordingly, when the opposite rotation takes place, an opposite passageway follows for the absorbent pads 20 into the holder 10.

The foremost absorbent pad 20 protrudes slightly out of the holder 10 so that the absorption layer 22 is available for use. A resistance element can be mounted in a pad of the absorbent pads 20 in or near the exit opening 13 to ensure the foremost pad cannot fall out prematurely and to create a discernable dispensing moment. Such a resistance can, for example, be realised through an edge in the opening 13 that goes inside or by raising the spiral groove 14 near the end thereof.

An extended side wall 26 has been provided in a second and third embodiment example of the absorbent pad to ensure that an edge of the holder does not come into contact with a s surface to treat when using the foremost absorbent pad, see FIGS. 6 and 7, that separates the bottom 25 of the pad from the absorbing top layer. These two further embodiment examples mainly distinguish themselves from each other due to their form and the embodiment of the absorption layer 22. Wing instruments 23 also protrude laterally here at the bottom from the absorbent pad to hold on to the spiral profile. As is the case for the first embodiment example, the absorbent pads can be stacked to increase their mutual packaging density. The side wall is conical for an important part of its height h and at least it has been widened for this purpose.

A ridge 24 that extends laterally and outwardly has been, moreover, provided in the embodiment examples of FIGS. 6 and 7 to receive upon it the bottom 25 of an absorbent pad stacked on it. Such a ridge 24 does not only boost the mutual discharge of the absorbent pads but it also avoids the height h thereof that has been selected to be larger than the thickness d of the absorption layer 22 that presses the top absorbent pad on to the absorption layer of an absorbent pad that is below. This is, in particular, beneficial if the absorption layer has been provided with an active liquid that, therefore, is not squeezed from it when different absorbent pads are held in the holder. Sealing is, moreover, achieved due to the accurate fit of the bottom 25 of an absorbent pad on the ridge 24 of an absorbent pad that lies below that will preserve the absorption layer from premature drying out as a result of evaporation.

The holder is, thus, in particular, suitable for use with absorbent pads that have been wetted in advance with an active liquid.

The device according to the invention can be realised for a relatively low cost price. FIG. 5 indicates through an exploded view that the device, including the sealing cap, is assembled from merely three relatively easy to produce injection moulded parts 10, 30, 40 using a suitable plastic, for example, a thermoplastic such as polyethylene, polypropylene or polystyrene. The parts can be assembled together after being formed to create the full device. The device according to the invention can, therefore, be realised in a special economic manner. The same applies to the absorbent pads 20 that consist of a relatively simple and inexpensive injection moulded basic part 21 to be manufactured on which a, for example, felt-, sponge- or cotton wool-type absorption layer 22 has been added. Adhesive pads 22 that have been prepared in advance are assumed as a benefit that are attached to fit on basic parts 21 within this context.

Although the invention was further explained based on merely a few embodiment examples, it should be clear that this does not limit the invention by any manner or means. On the contrary, there are still many variations and realisation forms that are possible within the framework of the invention for an average professional. For example, other materials may be selected for the applied materials to give the device an increased durability or luxury aspect. Further sealing means may also be mounted in the holder and in the absorbent pads to boost the shelf life of the absorption layer. Instead of a groove in a wall, the spiral profile may also comprise a helical edge on the wall in which case the holding instruments shall compromise complementary grooves. The propel means shall, in this case, exert an independent and separate hold on the absorbent pad. The design of, in particular, the outside of the device can also be adjusted and harmonised based on a special target group or use. In general, the invention can offer a refillable holder or a non-refillable holder for absorbent pads of the described type that is very effective when used and which, moreover, can be produced on a large and economic scale.

### REFERENCE NUMBERS

holder 10
storage chamber 11
wall 12
exit opening 13
spiral profile 14
absorbent pads 20
basic part 21
absorption layer 22
wing instruments 23
ridge 24
bottom 25
side wall 26
height h
thickness d
applicators 30
control instrument 31
pushing bodies 32
space 33
attachment clip 35
sealing cap 14

1. Device for retaining and dispensing in a controlled fashion at least partially porous absorbent pads, comprising an elongated storage chamber that is configured to receive therein a series of absorbent pads and comprising actuators that are configured to move an absorbent pad in the storage chamber to an exit opening and dispense it at the exit opening whereby the actuators comprise a manually controllable control instrument that is configured to enforce a rotation on the carrier bodies wherein the actuators comprise a spiral profile over at least nearly a full length of an internal wall of the storage chamber, the absorbent pads comprise holding means that are configured to work as an assembly together with the spiral profile in the storage chamber to enforce an absorbent pad a passage according to the spiral profile by the control instrument enforced rotation thereon, and the control instrument is configured to directly hold the absorbent pads.

2. Device according to claim 1 wherein the spiral profile comprises a groove that is mounted in the internal wall and the holding means comprise at least one wing instrument that protrudes to one side of an absorbent pad and is configured to enter into the spiral profile.

3. Device according to claim 1 wherein the control instrument comprises a rotation instrument mounted on an end opposite of the exit opening of the holder, and whereof at least one elongated propel instrument protrudes that extends over at least nearly a full length of the storage chamber at least nearly parallel to a spiral axis of the spiral profile and that is configured to hold the absorbent pads.

4. Device according to claim 3 wherein the at least one propel instrument extends between the stored absorbent pads and the internal wall of the storage chamber.

5. Device according to claim 3 wherein the propel instrument is configured to hold on to the holding means of the absorbent pads.

6. Device according to claim 3, wherein the absorbent pads each comprise a number of holding instruments regularly distributed over an outline of the absorbent pad and the control instrument comprises a similar number of propel instruments that are configured to hold the holding instruments and at least nearly fittingly extend between the holding instruments.

7. Device according to claim 6 wherein the actuators comprise three propel instruments that extend thereof with a mutual angle of approximately 120 degrees with regard to the spiral axis.

8. Device according to claim 1 wherein a resistance element is included in a path of the holding means near the exit opening.

9. Device according to claim 8 wherein the resistance element comprises at least a part of a peripheral edge that extends internally in the exit opening and behind to which a carrier element hooks at least temporarily before exiting through the exit opening.

10. Device according to claim 1 wherein the exit opening is sealable with a sealing cap.

11. Device according to claim 10 wherein the sealing cap, the holder body and the control instrument are at least externally cylindrical with a common external diameter.

12. Device according to claim 11 wherein at least the control instrument can be recognised as such.

13. Absorbent pad for application with the device according to claim 1, wherein the absorbent pad comprises an at least nearly form-retaining basic part that is provided on a topside with a porous absorption layer and holding means in the basic part that are configured to work together with a spiral profile on an internal sides of the device.

14. Device according to claim 13 wherein the absorption layer contains an active liquid.

15. Device according to claim 13 wherein the basic part is provided with the holding means at a bottom side and is limited between the bottom side and the topside by an extended side wall.

16. Device according to claim 13 wherein the basic part comprises a hollow body that is open on the bottom, of which a hole between the bottom side and the topside is limited by the raised side wall and the absorbent pad can be nested with a similar absorbent pad.

17. Device according to claim 16 wherein the side wall extends conically over part of a height thereof, at least extends running out.

18. Device according to claim 15 wherein the side wall comprises a ridge at the bottom side that is capable and adjusted to receive thereon a corresponding ridge of an absorbent pad that is stacked thereon and at the height of the ridge is provided with the holding means.

19. Device according to claim 18 wherein the ridge has a height that is greater than a thickness of the absorption layer.

20. Device according to claim 18 wherein the side wall seals vapour-tight on the ridge.
